(19) United States
(12) Patent Application Publication
(10) Pub. No.: US 2024/0393494 A1
(43) Pub. Date: Nov. 28, 2024

(54) MACHINE LEARNING-ASSISTED FULL-BAND INVERSION FOR BOREHOLE SENSING

(71) Applicant: Halliburton Energy Services, Inc., Houston, TX (US)

(72) Inventors: Xusong WANG, Singapore (SG); Xiang WU, Singapore (SG); Christopher Michael JONES, Houston, TX (US); Jichun SUN, Singapore (SG); Ahmed Elsayed FOUDA, Pearland, TX (US)

(73) Assignee: Halliburton Energy Services, Inc., Houston, TX (US)

(21) Appl. No.: 18/389,077

(22) Filed: Nov. 13, 2023

Related U.S. Application Data
(60) Provisional application No. 63/469,191, filed on May 26, 2023.

(51) Int. Cl. G01V 20/00; G16C 20/70

(52) U.S. Cl. CPC G01V 20/00; G16C 20/70

(57) ABSTRACT

The present disclosure provides techniques to identify material properties from measured physical response data in ways that are more efficient than conventional numerical inversion. Systems and techniques of the present disclosure may use machine learning (ML) techniques to generate mappings that map specific physical responses to specific material properties based on knowledge of factors that may be inherent to certain types of sensing equipment. Data generated by a ML computer model may be constrained, altered, or filtered to more closely correspond to characteristics known to be associated with a certain type of sensing equipment. Such a constrained model may identify fewer possible results as compared to an unconstrained model, thereby, solving problems that may be encountered when using ML techniques to identify material properties from measured responses.

This application claims benefit of U.S. Provisional Application No. 63/469,191 filed May 26, 2023, which is incorporated herein by reference.

### TECHNICAL FIELD

### Technical Field

The present disclosure is directed to making evaluations from data sensed in a wellbore. More specifically, the present disclosure is directed to constraining portions of computer modeled data when evaluations are made that compare the computer modeled data to data measured in a wellbore.

### Background

Tools are often employed in wellbore environments for a variety of purposes. In some instances, acoustic sensors may be deployed in a wellbore when evaluations are made relating to whether a wellbore is operation is proceeding properly. In other instances, electromagnetic sensors or nuclear magnetic resonance sensors may be deployed in a wellbore when determinations are made regarding structures or properties materials within the Earth are identified. Evaluations made using sensed data are important to making determinations regarding how a wellbore should be managed. The faster or more accurate that such determinations are made, may allow an operator of a wellbore to perform wellbore operations more quickly and efficiently.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1A is a schematic diagram of an example logging-while-drilling (LWD) environment, in accordance with various aspects of the subject technology.

FIG. 1B is a schematic diagram of an example wireline logging environment, in accordance with various aspects of the subject technology.

FIG. 2 illustrates actions that may be performed by a machine learning process that may be used to refine the machine learning process, in accordance with various aspects of the subject technology.

FIG. 3 illustrates other actions that may be performed by a machine learning process when the machine learning process is refined.

FIG. 4 illustrates a flow of actions that may be performed during a machine learning training stage and during a machine learning application stage, in accordance with various aspects of the subject technology.

FIG. 5 illustrates a series of actions that may be performed when a machine leaning computer model performs evaluations that compare measured data with data calculated or otherwise determined by the machine learning computer model, in accordance with various aspects of the subject technology.

FIG. 6 illustrates an example architecture of a computing device which can implement the various technologies and techniques described herein.

### DETAILED DESCRIPTION

In current borehole well logging process, for example, using tools that include acoustic, electromagnetic, and nuclear magnetic tools, inversion is one of the process that may be used to identify material properties that exist in a subterranean environment from data collected using these tools. Machine learning is an artificial intelligent technology that may be used to map an input to an output. When performing tasks in the oil & gas industry, machine learning may be used to substitute forward modelling that identifies a numerical solution, yet machine learning is not well suited to solving problems that inversion is commonly used to solve. What this means is that conventionally machine learning may be used to map from material properties to physical responses, yet machine learning has not been successful in mapping from physical responses to material properties. One reason for this is that attempts to use machine learning to map from physical responses to material properties is not stable because such a mapping does not result in a one-to-one mapping.

For a mapping of physical responses to material properties to be stable, a one-to-one correspondence of a physical property to a material property may have to be identified. Rules associated with such a one-to-one mapping may require that a computer model generate only one response that potentially corresponds to a measured physical response. Such a rule may specify a requirement for identifying when data from a machine learning process sufficiently maps physical responses to material properties. Such a requirement may include identifying that any errors associated with comparing the physical properties to the material properties are within a threshold level. One reason that machine learning processes may not be stable may relate to the fact that measured or sensed data and calculated data may not have compatible bandwidths. For example, data that includes sensed acoustic or vibration information may not include a band of frequencies below a first frequency or above a second frequency where data of a machine learning (ML) computer model may not be so constrained. This means that data identified by operation of a ML computer model may have a bandwidth that is larger than a bandwidth of measured data. Methods of the present disclosure may, therefore, compensate for such differences by constraining results generated by the ML computer model. This means that a bandpass or other filtering function may be performed to filter data generated by the ML computer model to match an expected bandwidth of the sensed data. This bandpass filtering function may suppress outputs of the ML computer model that are associated with frequencies lower than the first frequency and/or frequencies that are greater than the second frequency. Such a filtering process may be capable of generating a one-to-one mapping of physical responses to material properties in instances when without the filtering process, a resulting mapping is a one-to-many (1 to N) mapping.

A machine learning (ML) model, or ML computer model may be implemented using one or more processors, a network of computers, or as a neural network. This model operates based on algorithms that use data inputs and desired outputs for training. The ML model may engage in a recursive process, repeatedly conducting evaluations and updating factors included in a equation or coefficients of equations, based on the data and computations. This iterative process continues until the outcomes from the ML model converge to a resolution, effectively addressing the problem it has been tasked to solve. After that trained ML computer model could be deployed for application or prediction.

Other functions that may be performed to limit or update data identified using a ML computer model or to update the ML computer model is to constrain evaluations to one or more classifications of materials or material properties. Additionally or alternatively, calculations may be performed to identify gradients that may be used by or incorporated into the ML computer model. In such instances, one or more processors may execute instructions of the computer model such that functions or actions of the computer model can be performed.

One reason to replace conventional inversion with machine learning is to identify material properties of a wellbore more quickly than possible using the conventional inversion techniques. Sensing equipment that may be used to acquire data from which material properties are identified may be used to shorten the development process of a wellbore or may be used to improve the operation of the wellbore. By more quickly modeling subterranean structures one or more types of wellbore operations may be initiated more quickly. This may allow hydrocarbons to be extracted at scheduled rates, carbon dioxide (CO2) be sequestered according to a plan, or a hydraulic fracturing process to be implemented more efficiently. For example, an acoustic source may be used to generate images based on how acoustic energy is reflected back to a sensor or is absorbed by materials that are impacted by the acoustic energy. An ultrasonic imaging sensor array is an example of an acoustic sensing device. An example of an electromagnetic sensing device is a device that includes transmitting coils and reception coils.

No matter what type of sensing equipment is used, these tools are designed to make measurements that are converted into sets of acquired data. These sets of acquired data may be used as input to processes that identify physical and/or material properties of materials based on knowledge of physics and how the physical and/or material properties of the materials are affected by the sensing equipment.

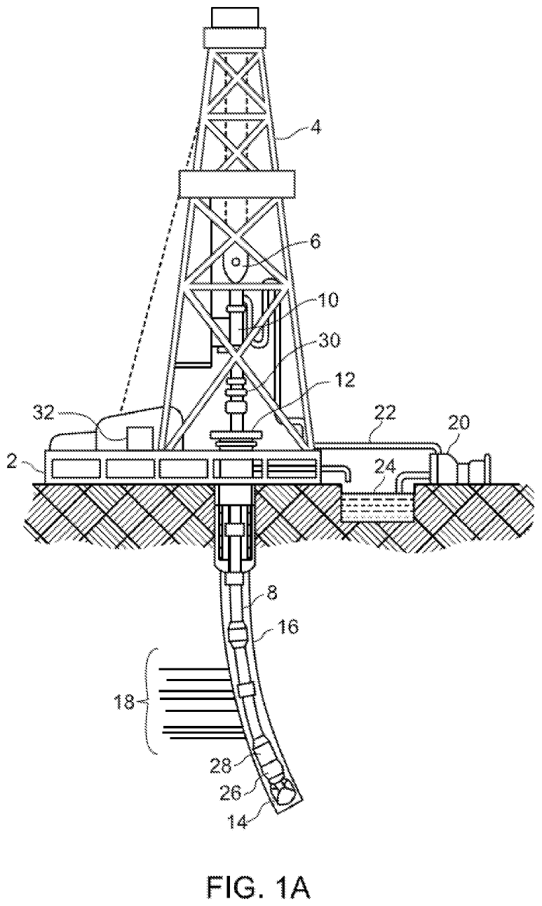

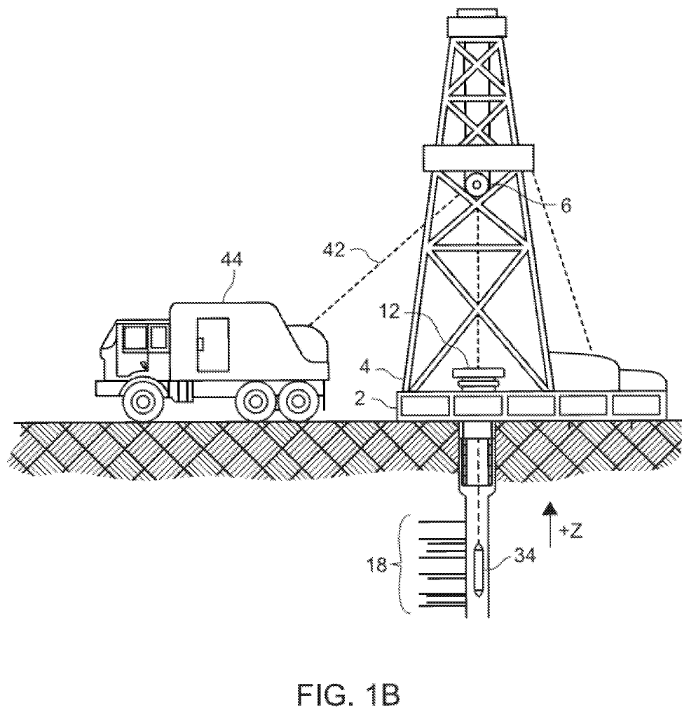

The disclosure now turns to FIGS. 1A-B that provide a brief introductory description of some systems that can be employed to practice the concepts, methods, and techniques disclosed herein. A more detailed description of the methods and systems for implementing the improved semblance processing techniques of the disclosed technology will then follow.

FIG. 1A shows an illustrative logging while drilling (LWD) environment. A drilling platform 2 supports derrick 4 having traveling block 6 for raising and lowering drill string 8. Kelly 10 supports drill string 8 as it is lowered through rotary table 12. Drill bit 14 is driven by a downhole motor and/or rotation of drill string 8. As bit 14 rotates, it creates a borehole 16 that passes through various formations 18. Pump 20 circulates drilling fluid through a feed pipe 22 to kelly 10, downhole through the interior of drill string 8, through orifices in drill bit 14, back to the surface via the annulus around drill string 8, and into retention pit 24. The drilling fluid transports cuttings from the borehole into pit 24 and aids in maintaining borehole integrity.

Downhole tool 26 can take the form of a drill collar (i.e., a thick-walled tubular that provides weight and rigidity to aid the drilling process) or other arrangements known in the art. Further, downhole tool 26 can include acoustic (e.g., sonic, ultrasonic, etc.) logging tools and/or corresponding components, integrated into the bottom-hole assembly near bit 14. In this fashion, as bit 14 extends the borehole through formations, the bottom-hole assembly (e.g., the acoustic logging tool) can collect acoustic logging data. For example, acoustic logging tools can include transmitters (e.g., monopole, dipole, quadrupole, etc.) to generate and transmit acoustic signals/waves into the borehole environment. These acoustic signals subsequently propagate in and along the borehole and surrounding formation and create acoustic signal responses or waveforms, which are received/recorded by evenly spaced receivers. These receivers may be arranged in an array and may be evenly spaced apart to facilitate capturing and processing acoustic response signals at specific intervals. The acoustic response signals are further analyzed to determine borehole and adjacent formation properties and/or characteristics.

For purposes of communication, a downhole telemetry sub 28 can be included in the bottom-hole assembly to transfer measurement data to surface receiver 30 and to receive commands from the surface. Mud pulse telemetry is one common telemetry technique for transferring tool measurements to surface receivers and receiving commands from the surface, but other telemetry techniques can also be used. In some embodiments, telemetry sub 28 can store logging data for later retrieval at the surface when the logging assembly is recovered.

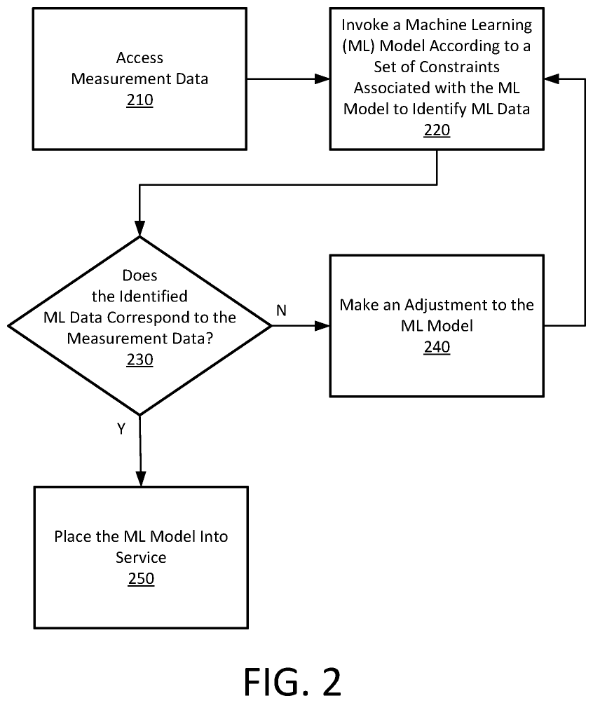

At the surface, surface receiver 30 can receive the uplink signal from the downhole telemetry sub 28 and can communicate the signal to data acquisition module 32. Module 32 can include one or more processors, storage mediums, input devices, output devices, software, and the like as described in detail in FIGS. 2A and 2B. Module 32 can collect, store, and/or process the data received from tool 26 as described herein.

FIG. 1B illustrates how a tool used to collect data may be lowered down a wellbore. FIG. 1B includes many of the same element discussed in respect to FIG. 1B. For example, FIG. 1B includes platform 2, derrick, 4, block 6, and rotary table 12 that are included in FIG. 1A. At various times during the drilling process, drill string 8 shown in FIG. 1A may be removed from the borehole and downhole tool 34 may then be lowered into the wellbore 16 of FIG. 1A. Once drill string 8 has been removed, logging operations can be conducted using a downhole tool 34 (i.e., a sensing instrument sonde) suspended by a conveyance 42. In one or more embodiments, the conveyance 42 can be a cable having conductors for transporting power to the tool and telemetry from the tool to the surface. Downhole tool 34 may have pads and/or centralizing springs to maintain the tool near the central axis of the borehole or to bias the tool towards the borehole wall as the tool is moved downhole or uphole.

Downhole tool 34 can include an acoustic or sonic logging instrument that collects acoustic logging data within the borehole 16. A logging facility 44 includes a computer system, that may be used to collect, store, and/or process measurements gathered by logging tool 34. In one or more instances, conveyance 42 may include at least one of wires, conductive or non-conductive cable (e.g., slickline, etc.) coupled to downhole tool 34. Conveyance 42 may include tubular conveyances, such as coiled tubing, pipe string, or a downhole tractor. The downhole tool 34 may have a local power supply, such as batteries, a downhole generator, or the like. When employing non-conductive cable, coiled tubing, pipe string, or downhole tractor, communication can be supported using, for example, wireless protocols (e.g. EM, acoustic, etc.), and/or measurements and logging data may be stored in local memory for subsequent retrieval.

Downhole tool 34 may include one or more of a hydrophone, a microphone, an array of hydrophones, or an array of microphones. Such arrays may include one or more hydrophones and/or hydrophones that collect data from a wellbore at various stages of the wellbores life span, from initial phases where wellbores are drilled and made, to when the wellbore is used during a production process (e.g., hydrocarbon extraction or carbon sequestration process), and/or to after a wellbore is put out of service.

Although FIGS. 1A and 1B depict specific borehole configurations, it is understood that the present disclosure is equally well suited for use in wellbores having other orientations including vertical wellbores, horizontal wellbores, slanted wellbores, multilateral wellbores and the like. While FIGS. 1A and 1B depict an onshore operation, it should also be understood that the present disclosure is equally well suited for use in offshore operations. Moreover, the present disclosure is not limited to the environments depicted in FIGS. 1A and 1B, and can also be used, for example, in other well operations such as production tubing operations, jointed tubing operations, coiled tubing operations, combinations thereof, and the like.

The scope of the present disclosure is not limited to the environment shown in FIGS. 1A and 1B as methods of the present disclosure may be applied in other environments. Methods and apparatus of the present disclosure may process acoustic data that was received from one or more microphones, hydrophones, piezoelectric sensors, or other equipment that may be capable of sensing acoustic signals, such as sub-sonic, sonic, or ultrasonic signals. This processing may include performing evaluations that allow portions of received acoustic data to be identified based on characteristics known to be representative of specific types of sound sources. Characteristics that may be associated with a sound source include yet are not limited to one or more frequencies emitted by the sound source and/or information that can be used to identify a location of the sound source. Additionally, or alternatively, acoustic noise characteristic of a sound source may be associated with a sound amplitude, a power, or a power spectral density of the noise emitted by the sound source.

FIG. 2 illustrates actions that may be performed by a machine learning process that may be used to refine the machine learning process. At block 210 measurement data may be accessed. This measurement data may have been collected from a wellbore using any type of sensing equipment known in the art. As such, the measurement data accessed at block 210 may be acquired using an acoustic tool, an electromagnetic imaging tool, a nuclear magnetic resonance tool, or other type of tool. At block 220, a machine learning (ML) computer model may be invoked according to a set of constraints. This may allow the ML computer model to generate ML data that may correspond to measured or sensed data such that materials included in the wellbore may be identified.

The operation of a ML computer model may be constrained to generate ML data that has a particular frequency response. This may result in the ML computer model filtering out or otherwise suppressing frequency components that do not correspond to a frequency response known to be associated with a particular type of wellbore sensing equipment or materials located in a wellbore. For example, a filtering function may remove frequencies from a set of computer generated data that are below a first frequency and above second frequency based on knowledge of frequencies that are not consistent with a type of sensing equipment.

Alternatively or additionally, the constraints of the ML computer model may result in the ML computer model making evaluations based on a type or class of material that exists in the wellbore. This may mean that evaluations may be limited to a class or type of materials that are assumed be in or have been shown to exist in the wellbore. For example, reflections characteristics of the wellbore may be consistent with sandstone of a given porosity and this characteristic may allow the ML computer model to limit evaluations to a set of constraints based on a rule associated with sandstone and that given porosity. These constraints may include identifying the first and the second frequency of a bandpass filter like the filter discussed above, or may include identifying other constraints known to be associated with sandstone.

Determination block 230 may then identify whether the ML data at block 220 corresponds to the measurement data to a threshold level. This threshold level may require that a measured parametric or value be within a certain percentage of a value of that same parametric or value generated by operation of the ML computer model. For example, when an attenuation forecast by the ML computer model identifies that an attenuation of acoustic or electromagnetic signal should have a value of 50 decibels, that a measured value of that attenuation should be within 50 decibels plus or minus ten percent. When determination block 220 identifies that the ML data does not correspond to the measurement data within the threshold level, program flow may move to block 240 where the ML computer model is updated. The ML computer model may then again be invoked at block 220 using the updated ML computer model. At least some of the actions performed in FIG. 2 may be performed iteratively until the ML data generated by the ML computer model corresponds to the measurement data to the threshold level (i.e., matches to a threshold degree). This may allow for multiple evaluations to be performed with different constraints or coefficients of formulas associated with identifying materials. For example, sets of constraints may be associated with a frequency bandpass function or with particular types of materials or material characteristics or responses. These constraints may also correspond to material properties modeled by the ML computer model. As such, blocks 220, 230, and 240 may be used to generate or choose one or more ML algorithms based on what may be referred to as a learning process.

When determination block 230 identifies that the ML data corresponds to the measurement data, program flow may move to block 250 where the ML computer model is placed into service. This means that determinations made by the ML computer model may be relied upon when decisions about the wellbore are made in an actual ML application. For example, the ML computer model may identify that the material properties of a location within a subterranean formation are suitable for hydrocarbon extraction or the sequestration of carbon dioxide (CO2). Based on this determination, either an automated process or an operator of the wellbore may authorize that the hydrocarbons be extracted from or that CO2 should be sequestered to the subterranean formation. Methods of the present disclosure, therefore, allow ML learning to replace conventional inversion techniques and may use measured or sensed physical responses such that material properties can be identified more efficiently.

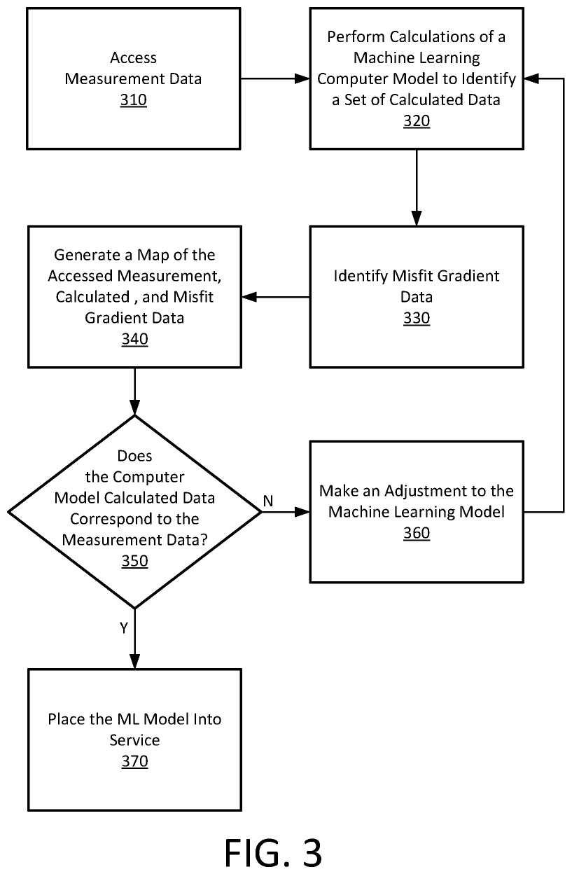

FIG. 3 illustrates other actions that may be performed by a machine learning process when the machine learning process is refined. At block 310 measurement data may be accessed as discussed in respect to block 210 of FIG. 2. One or more processors may then execute instructions of an ML computer model to identify a set of calculated data at block 320. This may include applying the constraints discussed in respect to the actions of FIG. 2. This set of calculated data may be bandwidth limited in a manner similar to the bandpass filter discussed above or this calculated data may be used to identify material property or type of material located in the wellbore.

At block 330 misfit gradient data may be calculated. This may include preforming calculations according to formula 1 and 2 below. In formulas 1 through 5 below, Σ represents a summation function, F (m) and F (mᵢ₋₁) represent functions of model m associated with different values, d is data collected by sensors, S is a fitness estimation between a current model and prior known (prior knowledge) information, λ is a weighting factor, mᵢ is a value of the model m at a particular update, mᵢ₋₁ is value of the model m immediately before the particular update, σ is an update step along a gradient inverse direction, ∇ represents a gradient function, and F′(mᵢ₋₁) is a derivative referred to as a forward modeling gradient function.

$$Error = Cost Function Value = {Σ ( d - F ( m ) )}^{2} + Prior Knowledge \qquad Formula 1$$

$$Error = Cost Function Value = {Σ ( d - F ( m ) )}^{2} + λ S ( m ) \qquad Formula 2$$

$${m}_{i} := {m}_{i - 1} - σ ( ∇ [ {Σ ( d - F ( {m}_{i - 1} ) )}^{2} + λ S ( {m}_{i - 1} ) ] \qquad Formula 3$$

$${m}_{i} := mi - 1 - σ ( ∇ [ {Σ ( d - F ( {m}_{i - 1} ) )}^{2} + ∇ λ S ( {m}_{i - 1} ) ] ) \qquad Formula 4$$

$$( ∇ [ {Σ ( d - F ( {m}_{i - 1} ) )}^{2} ] ) = - 2 [ {F}^{′} ( {m}_{i - 1} ) ( d - F ( {m}_{i - 1} ) ) ] \qquad Formula 5$$

Formulas 1 and 2 may be referred to as a machine learning assisted or artificial intelligence (AI) assisted inversion and may be used to map a model space of function F (m) to a data space. As such, an error or cost function may be identified using measured data d and ML model data of function F (m). This error or cost function may be calculated based on prior knowledge that may be expressed as AS (m).

Such Prior knowledge may be general knowledge about a particular material. For example, in seismic inversion, it may be assumed the reflectivity is sparse or limited to certain dimensions. The reason why such an assumption may be made is that recorded seismic waves may have a narrow bandwidth (limited frequency response), however, the earth reflectivity may have a larger or full bandwidth. To fill a gap of high frequency and low frequency loss associated with measured data, the sparsity assumption to may be used to make an inversion stable by limiting computer modeled data to the high and low frequency loss characteristics assumed to be associated with the measured data. A formula λ∥r_{M}x1∥₁ may be used where A is a prior knowledge coefficient, r is the earth reflectivity, its dimension may be Mx1; ∥r_{M}x1∥₁ is the earth reflectivity normal level.

In borehole sensor physics, such as borehole detection, we also have many sets of prior knowledge. For example, when acoustic measurements are used, we know the impedance between borehole and a pipe should correspond to a step function. But we may not know how strong an impedance contrast is. Methods of the present disclosure may have to take this into account when prior knowledge is used to perform computer modeled inversions.

In another example, an electromagnetic wave may be used to detect pipe erosion. In such an instance, we may roughly know the pipe permeability and resistivity and what may be unknown is the pipe thickness due to metal loss or metal gain. So, during inversion, we can set the permeability and resistivity range as a guide to stabilize the inversion and get a reasonable pipe thickness. In summary, the prior knowledge used is a case-by-case estimate, that may need to be customized. This may require changing a mathematical form to accommodate the prior knowledge such that inversion workflow can be expected to yield reliable results.

Values of model m (e.g., mᵢ and mᵢ₋₁) may be calculated according to the formula 3 or 4 above. Equation ∇[Σ(d−F (mᵢ₋₁))²] may be referred to as the misfit gradient equation and equation ∇λS(mᵢ₋₁) may be referred to as the prior knowledge constraint gradient equation. Classical methods may be used to calculate values of function F(mᵢ₋₁) such that the misfit gradient equation may be used to calculate values. Machine learning may be used to replace techniques associated with traditional inversion. Machine learning or AI may thus be used to replace numerical solutions identified using classical numerical forward modeling and/or classical solutions that identify data misfit gradients. Formula 5 is a simplified data misfit gradient equation. Formula 5 may be used to simplify calculations performed by a machine learning process.

At block 340 a mapping of measurement data accessed at block 310, the data calculated at block 320, and the misfit gradient data identified at block 330 may be generated. Determination block 350 may then identify whether the data calculated by the ML computer model corresponds to the measurement data, when no, program flow may move to block 360 where an adjustment may be made to the ML computer model. Blocks 320, 330, 340, and 350 may be performed iteratively until determination block 350 identifies that the data calculated by the ML computer model corresponds to the measurement data such that the ML computer model may be placed into service at block 370. As discussed in respect to FIG. 2, placing the ML computer model into service may allow determinations made by the ML computer model to be relied upon when decisions about the wellbore are made. Here again, this may mean that the ML computer model may be relied upon to identify locations where material properties of a subterranean formation are suitable for hydrocarbon extraction, a CO2 sequestration process, or some other wellbore process.

In regard to the Latent space discussed below, when working with data that is especially complex and high-dimensional. For example, data that has the complexity of images or audio files may not be not easy to work with directly as it may contain a lot of unnecessary information. Additionally or alternatively, it might be hard to find patterns in this data that are useful to a particular evaluation.

The term “Latent” essentially means “hidden” or “underlying”. So, the Latent space represents the underlying or hidden features that are most important in our data. Imagine looking at a bunch of photos of people's faces. To a computer, these photos are just a big array of numbers (pixels). But to people, there are certain important features that distinguish one face from another, such as the shape of the eyes, the size of the nose, the color of the hair, etc. The Latent space is a way of transforming the raw data (the pixels in this case) into a simpler, more compact representation that captures these important features. In this transformed space, similar faces will be close together and different faces will be farther apart. This makes it much easier to work with the data, because we can operate on the important features directly, instead of having to deal with every single pixel individually.

In machine learning, the model may learn to automatically extract these important features and represent the data in the Latent space. The process of learning this transformation is typically done using a method called “training” where the model learns from a large amount of example data. Once the model is trained, it can map new data into the latent space, even if it's never seen that exact data before.

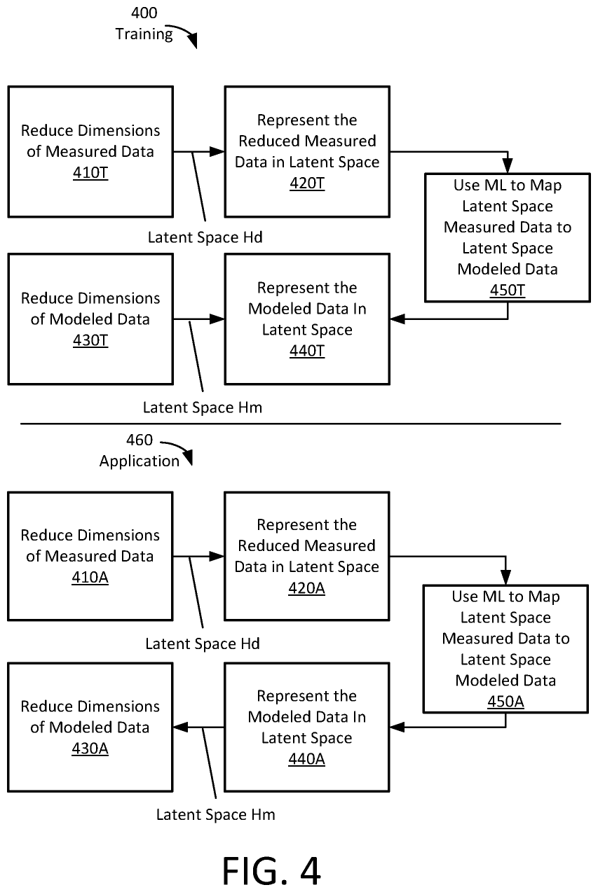

FIG. 4 illustrates a flow of actions that may be performed during a machine learning training stage and during a machine learning application stage. A first flow of actions 400 shown in FIG. 4 includes blocks 410T, 420T, 430T, and 450T may be implemented with an ML computer model is trained. At block 410T, measured data d acquired from a wellbore may be reduced in dimensions. Such a reduction of dimensions may include mapping three-dimensional (3D) data into two dimensions (2D), for example. By reducing the number of dimensions associated with a set of measured data d, calculations or other evaluations performed based on that calculated data may be simplified. At block 420T the measured data d mapped in block 410 may be represented in Latent space of measured data Hd, for example, in a 2D mapping. At block 430T a set of data generated based on operation of an ML computer model m may be reduced in dimensions. This may include mapping data identified by operation of an ML computer model in 3D space to a 2D space. At block 440T, the ML computer model data m mapped at block 430T may be represented in the Latent space of modeled data Hm. For example, here again in the 2D space. At block 450 an ML process may be used to map the Latent space measured data Hd to the Latent space modeled data Hm. This process may be performed iteratively and may be performed after any of the actions discussed in respect to FIGS. 2-3 have been performed. Once errors associated with differences between the measured data and the ML computer modeled data have been reduced to the threshold level, training stage 400 may be determined to be complete and operation of the ML computer model may move to the application stage.

A second flow of action 460 shown in FIG. 4 show blocks 410A, 420A, 430A, 440A, and 450A may be implemented when the ML computer model is used to identify material properties of a wellbore from measured data. Here again a number of dimensions of a set of measure data may be reduced at block 410A (e.g., from a 3D representation to a 2D representation). That reduced set of measured data may be represented in Latent space (e.g., the 2D space) and the Latent space measured data Hd may be mapped to operations of the ML computer model at block 450. At block 440A the ML computer model may be configured to represent ML computer modeled data in the Latent space Hm and the dimension reduction performed at block 430A may reconstruct data m associated with the ML computer model in Latent space Hm.

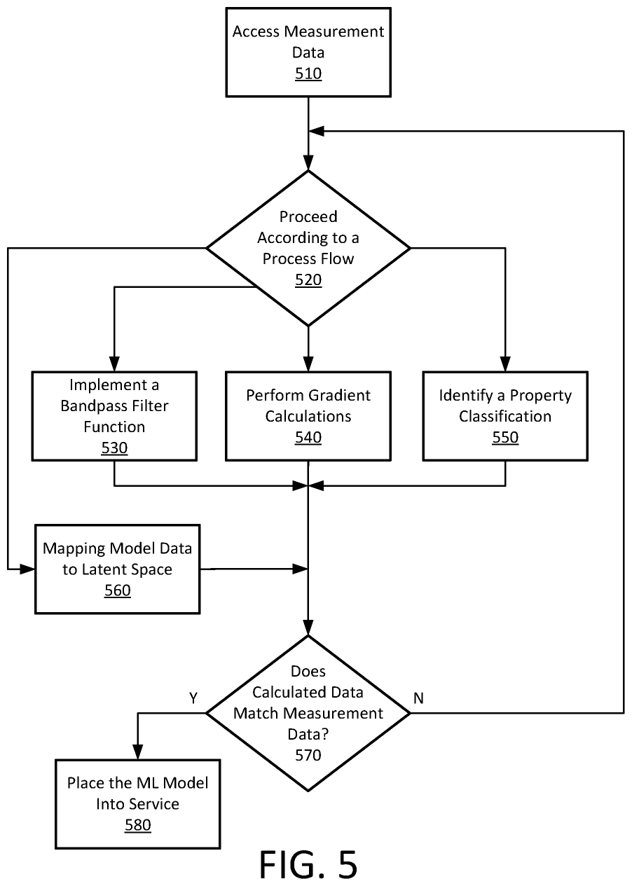

FIG. 5 illustrates a series of actions that may be performed when a machine leaning computer model performs evaluations that compare measured data with data calculated or otherwise determined by the machine learning computer model. At block 510 measurement data sensed at a wellbore may be accessed. Determination block 520 may identify a process flow that should be used when determinations of the present disclosure are made. This means that actions of either of blocks 530, 540, 550, or 560 may be performed. A process flow could potentially include an ML computer model making calculations. A calculated dataset may then be filtered using a filter (e.g., a bandpass) filter at block 530. Alternatively or additionally, this process may include performing gradient calculations at block 540 and/or identifying a property classification at block 550. At block 560 ML computer model data may be mapped to a latent space when the ML computer model is trained as discussed in respect to FIG. 4. Determination block 570 may then identify whether calculated data matches measurement data to a threshold degree or level, when no, program flow may move back to determination block 520. When determination block 570 identifies that the calculated data matches the measurement data, the ML computer model may be placed into service at block 580.

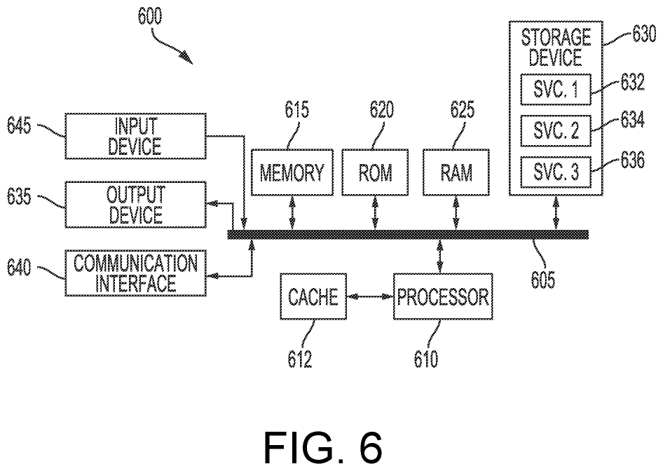

FIG. 6 illustrates an example architecture 600 of a computing device which can implement the various technologies and techniques described herein. The various implementations will be apparent to those of ordinary skill in the art when practicing the present technology. Persons of ordinary skill in the art will also readily appreciate that other system implementations or examples are possible. The components of the computing device architecture 600 are shown in electrical communication with each other using a connection 605, such as a bus. The example computing device architecture 600 includes a processing unit (CPU or processor) 610 and a computing device connection 605 that couples various computing device components including the computing device memory 615, such as read only memory (ROM) 620 and random-access memory (RAM) 625, to the processor 610.

The computing device architecture 600 can include a cache of high-speed memory connected directly with, in close proximity to, or integrated as part of the processor 610. The computing device architecture 600 can copy data from the memory 615 and/or the storage device 630 to the cache 612 for quick access by the processor 610. In this way, the cache can provide a performance boost that avoids processor 610 delays while waiting for data. These and other modules can control or be configured to control the processor 610 to perform various actions. Other computing device memory 615 may be available for use as well. The memory 615 can include multiple different types of memory with different performance characteristics. The processor 610 can include any general-purpose processor/multi-processor and a hardware or software service, such as service 1 632, service 2 634, and service 3 636 stored in storage device 630, configured to control the processor 610 as well as a special-purpose processor where software instructions are incorporated into the processor design. The processor 610 may be a self-contained system, containing multiple cores or processors, a bus, memory controller, cache, etc. A multi-core processor may be symmetric or asymmetric.

To enable user interaction with the computing device architecture 600, an input device 645 can represent any number of input mechanisms, such as a microphone for speech, a touch-sensitive screen for gesture input, keyboard, mouse, motion input, speech and so forth. An output device 635 can also be one or more of a number of output mechanisms known to those of skill in the art, such as a display, projector, television, speaker device, etc. In some instances, multimodal computing devices can enable a user to provide multiple types of input to communicate with the computing device architecture 600. The communications interface 640 can generally govern and manage the user input and computing device output. There is no restriction on operating on any particular hardware arrangement and therefore the basic features here may easily be substituted for improved hardware or firmware arrangements as they are developed.

Storage device 630 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 625, read only memory (ROM) 620, and hybrids thereof. The storage device 630 can include services 632, 634, 636 for controlling the processor 610. Other hardware or software modules are contemplated. The storage device 630 can be connected to the computing device connection 605. In one aspect, a hardware module that performs a particular function can include the software component stored in a computer-readable medium in connection with the necessary hardware components, such as the processor 610, connection 605, output device 635, and so forth, to carry out the function.

For clarity of explanation, in some instances the present technology may be presented as including individual functional blocks including functional blocks comprising devices, device components, steps or routines in a method embodied in software, or combinations of hardware and software.

In some instances the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can include, for example, instructions and data which cause or otherwise configure a general-purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can include hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific embodiments thereof, but those skilled in the art will recognize that the application is not limited thereto. Thus, while illustrative embodiments of the application have been described in detail herein, it is to be understood that the disclosed concepts may be otherwise variously embodied and employed, and that the appended claims are intended to be construed to include such variations, except as limited by the prior art. Various features and aspects of the above-described subject matter may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the method, algorithms, and/or operations described above. The computer-readable data storage medium may form part of a computer program product, which may include packaging materials.

The computer-readable medium may include memory or data storage media, such as random-access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

The various embodiments described above are provided by way of illustration only and should not be construed to limit the scope of the disclosure. For example, the principles herein apply equally to optimization as well as general improvements. Various modifications and changes may be made to the principles described herein without following the example embodiments and applications illustrated and described herein, and without departing from the spirit and scope of the disclosure. Claim language reciting “at least one of” a set indicates that one member of the set or multiple members of the set satisfy the claim.

### Aspects of the Disclosure

Aspect 1: A method of the present disclosure may include accessing wellbore measurement data; identifying by a machine learning process data that corresponds to the wellbore measurement data; implementing a function that constrains the machine learning process data when mapping the constrained machine learning process data to the wellbore measurement data; comparing the constrained machine learning process data to the wellbore measurement data; and identifying based on the comparison that the wellbore measurement data corresponds to the constrained machine learning process data based on an error being less than an error threshold.

Aspect 2: The method of Aspect 1, wherein the function that constrains the machine learning process data is implemented based on an assumption regarding the wellbore measurement data.

Aspect 3: The method of Aspect 1 or 2, wherein the function that constrains the machine learning process data extracts features from the machine learning process data.

Aspect 4: The method of any of Aspects 1 through 3, further comprising classifying material properties of the machine learning process data by narrowing an output range associated with the machine learning process data.

Aspect 5: The method of Aspect 4, further comprising generating a mapping of a space that associates the constrained machine learning process data with known wellbore properties.

Aspect 6: The method of Aspect 4, further comprising performing calculations to identify an error value by: subtracting values forecasted data identified by operation of a computer model from portions the wellbore measurement data to create a set of difference values; squaring the each of the set of difference values; and generating a sum of the set of squared difference values.

Aspect 7: The method of Aspect 6, wherein the generated sum also includes a set of weighted fitness estimate values.

Aspect 8: The method of Aspect 6, further comprising iteratively applying a data misfit gradient equation and a prior knowledge constraint equation to identify the values of the forecasted data.

Aspect 9: The method of Aspect 6, further comprising generating a mapping of a space that associates the constrained machine learning process data with known wellbore properties.

Aspect 10: The method of Aspect 4, further comprising performing calculations to identify an error value by: subtracting values forecasted data identified by operation of a computer model from portions the wellbore measurement data to create a set of difference values; squaring the each of the set of difference values; and generating a sum of the set of squared difference values.

Aspect 11: The method of Aspect 10, wherein the generated sum also includes a set of weighted fitness estimate values.

Aspect 12: The method of Aspect 10, further comprising iteratively applying a data misfit gradient equation and a prior knowledge constraint equation to identify the values of the forecasted data.

Aspect 13: The method of any of Aspects 1 through 12, wherein the function that constrains the machine learning process data limits a bandwidth associated with a portion of the wellbore measurement data.

Aspect 14: The method of Aspect 13, wherein the bandwidth is limited by applying a bandpass filter on the portion of the wellbore measurement data.

Apparatus of the present disclosure may include one or more sensors, memory, and one or more processors that execute instructions out of the memory to perform any of the Aspects of the disclosure discussed above. Furthermore, any of the Aspects discussed above may be implemented by a non-transitory computer-readable storage medium where one or more processors execute instructions out of the memory.

What is claimed is:

1. A method comprising:
accessing wellbore measurement data;
identifying by a machine learning process data that corresponds to the wellbore measurement data;
implementing a function that constrains the machine learning process data when mapping the constrained machine learning process data to the wellbore measurement data;
comparing the constrained machine learning process data to the wellbore measurement data; and
identifying based on the comparison that the wellbore measurement data corresponds to the constrained machine learning process data based on an error being less than an error threshold.

2. The method of claim 1, wherein the function that constrains the machine learning process data is implemented based on an assumption regarding the wellbore measurement data.

3. The method of claim 1, wherein the function that constrains the machine learning process data extracts features from the machine learning process data.

4. The method of claim 3, further comprising classifying material properties of the machine learning process data by narrowing an output range associated with the machine learning process data.

5. The method of claim 4, further comprising:
generating a mapping of a space that associates the constrained machine learning process data with known wellbore properties.

6. The method of claim 4, further comprising:
performing calculations to identify an error value by:
subtracting values forecasted data identified by operation of a computer model from portions the wellbore measurement data to create a set of difference values;
squaring the each of the set of difference values; and
generating a sum of the set of squared difference values.

7. The method of claim 6, wherein the generated sum also includes a set of weighted fitness estimate values.

8. The method of claim 6, iteratively applying a data misfit gradient equation and a prior knowledge constraint equation to identify the values of the forecasted data.

9. The method of claim 6, further comprising:
generating a mapping of a space that associates the constrained machine learning process data with known wellbore properties.

10. The method of claim 4, further comprising:
performing calculations to identify an error value by:
subtracting values forecasted data identified by operation of a computer model from portions the wellbore measurement data to create a set of difference values;
squaring the each of the set of difference values; and
generating a sum of the set of squared difference values.

11. The method of claim 10, wherein the generated sum also includes a set of weighted fitness estimate values.

12. The method of claim 10, iteratively applying a data misfit gradient equation and a prior knowledge constraint equation to identify the values of the forecasted data.

13. The method of claim 1, wherein the function that constrains the machine learning process data limits a bandwidth associated with a portion of the wellbore measurement data.

14. The method of claim 13, wherein the bandwidth is limited by applying a bandpass filter on the portion of the wellbore measurement data.

15. A non-transitory computer-readable storage medium having embodied thereon instructions that when executed by one or more processor result in the one or more processors:
accessing wellbore measurement data;
identifying by a machine learning process data that corresponds to the wellbore measurement data;
implementing a function that constrains the machine learning process data when mapping the constrained machine learning process data to the wellbore measurement data;
comparing the constrained machine learning process data to the wellbore measurement data; and
identifying based on the comparison that the wellbore measurement data corresponds to the constrained machine learning process data based on an error being less than an error threshold.

16. The non-transitory computer-readable storage medium of claim 15, wherein the function that constrains the machine learning process data is implemented based on an assumption regarding the wellbore measurement data.

17. The non-transitory computer-readable storage medium of claim 15, wherein the function that constrains the machine learning process data extracts features from the machine learning process data.

18. The non-transitory computer-readable storage medium of claim 17, further comprising classifying material properties of the machine learning process data by narrowing an output range associated with the machine learning process data.

19. The non-transitory computer-readable storage medium of claim 18, wherein the one or more processors execute the instructions to:
generate a mapping of a space that associates the constrained machine learning process data with known wellbore properties.

20. An apparatus comprising:
a memory; and
one or more processors that execute instructions out of the memory to:
access wellbore measurement data;
identify by a machine learning process data that corresponds to the wellbore measurement data,
implement a function that constrains the machine learning process data when mapping the constrained machine learning process data to the wellbore measurement data,
compare the constrained machine learning process data to the wellbore measurement data, and
identify based on the comparison that the wellbore measurement data corresponds to the constrained machine learning process data based on an error being less than an error threshold.
